(19) United States
(12) Patent Application Publication
(10) Pub. No.: US 2017/0049493 A1
(43) Pub. Date: Feb. 23, 2017

(54) SHOE FOR HELPING TO POSITION AND FIX A SURGICAL IMPLANT

(71) Applicant: IN2BONES, Ecully (FR)

(72) Inventors: Bertrand Xavier François Gauneau, Dardilly (FR); Eric Stéphane Fourcault, Ecully (FR); Jean-Christophe Alain Giet, Lyon (FR)

(73) Assignee: IN2BONES, Ecully (FR)

(21) Appl. No.: 15/240,551

(22) Filed: Aug. 18, 2016

(30) Foreign Application Priority Data
Aug. 19, 2015 (FR) FR-15 57811

(51) Int. Cl. A61B 17/88; A61B 17/80

(52) U.S. Cl. CPC A61B 17/8872; A61B 17/8061; A61B 2017/681

(57) ABSTRACT

The invention relates to a shoe (1) for helping to position and fix a surgical implant which is intended to be attached to osseous bodies of a patient, for example those of the forearm, said shoe (1) having a main body (24) and being characterized in that said main body (24) has:
at least one radiolucent zone (25) and at least one radiopaque zone (26), and
means (27) for positioning said shoe (1) with respect to the surgical implant in a predetermined relative position,
so as to allow monitoring of the positioning of said implant with respect to said osseous bodies of the patient.

Devices for helping to fit surgical osteosynthesis implants in place.

The present invention relates generally to the field of devices for helping to fit surgical osteosynthesis implants in place, particularly those designed for the treatment of fractures or breaks of osseous bodies situated in particular in the forearm.

The present invention relates more particularly to a shoe for helping to position and fix a surgical implant which is intended to be attached to osseous bodies of a patient, for example those of the forearm, said shoe having a main body.

The present invention also relates to a surgical kit that includes such a shoe.

To improve the reconstruction of bones, for example the bones of the forearm of a patient following certain types of fractures or breaks of the distal end of the radius, it is known to use a surgical implant, generally designated by the term “radial plate” in the case of treatment of a radius. This type of surgical implant is most often formed, or always formed, by a curved plate made of metal (Ti6Al4V, CrCo, etc.), such that the anterior surface of the plate has a convex shape matching the shape of the end portion of the radius that is to be repaired. The radial plate is intended to be fixed by screwing to at least two parts of the radius that have been separated by a fracture, in such a way that said plate straddles the one or more fracture lines and immobilizes said at least two parts of the radius with respect to each other and thereby promotes their fusion.

When, during a surgical operation, the surgeon proceeds to position the implant opposite the osseous bodies that are to be fused, he has to take care that said implant is positioned perfectly opposite the one or more facture lines.

Moreover, he has to take particular care to ensure that the screws, when implanted, are not trans-articular, that is to say they do not exit from the opposite face of the osseous body in which they are fixed, with the risk of said screws disturbing the correct function of the articulation of the bone with respect to the surrounding bones.

On account of the huge variety seen in the morphologies of the radius of patients, the latter in some cases tend to suffer pain during the osteosynthesis process. Sometimes, the rubbing of the tissues on the implant, especially on the edges of the latter, can also be a source of pain, despite the small thickness of the implant.

In particular, in the present example concerning the treatment of fractures or breaks of the distal end of the radius, the surgeon also has to be particularly careful about the positioning of the osteosynthesis plate opposite a very specific anatomical zone of the radius, known in the profession as the “watershed” line.

This “watershed” line may be considered in substance as the crest line of the palmar part of the radius, on each side of which the bone is lower down, and on which the tendons rub under tension.

It has been observed that, when the osteosynthesis plate is incorrectly positioned such that it projects past said “watershed” line, this causes problematic irritation of the surrounding flexor tendons. It is therefore strongly recommended to surgeons that they take care to position the osteosynthesis plate with precision so as to avoid the plate extending past this “watershed” line.

Traditionally, the surgeon in charge of fitting such a plate first of all positions the latter manually, with or without a special tool, in relation to the fracture line or fracture zone.

Then, in order to ensure that the screws for fixing said plate are oriented correctly through the osseous bodies, the surgeon usually uses a device called a shoe, which he places in contact with the free surface of the plate.

Such a shoe in fact has oblong holes, which are intended to be placed in a predetermined position opposite the drilled bores for fixation of the plate. These oblong holes are configured so as to permit, in a first stage, an angular limitation of the positioning of a bone-drilling guide, then, in a second stage, once the bone has thus been drilled in a desired orientation, to guide the placement of the screws for fixing the plate to the osseous bodies, so as to allow easy and precise positioning of these fixation screws according to said desired orientation.

The surgeon then uses X-rays to check the position of said plate with respect to the “watershed” line and, if need be, makes repeated adjustments.

Once the plate has been positioned and fastened to the osseous bodies by the fixation screws put in place with the aid of the shoe, the surgeon also uses X-rays to check the correct configuration of said screws.

Finally, the surgeon detaches the shoe from the plate and withdraws it from the body of the patient, since said shoe is not intended to remain implanted there.

The metallic composition of the known radial plates, like that of the fixation screws traditionally used, makes them in fact radiopaque, that is to say visible to X-rays by contrast with the biological environment in which they are implanted. Monitoring the position of the implanted plate with respect to the “watershed” line and monitoring the orientation of the fixation screws is thus made easier.

Although the known solutions using metallic osteosynthesis plates are generally satisfactory, it should be noted that the radiopacity of these plates not only prevents visualization of the fracture lines once the plate has been implanted in the patient, it also means that the screws for fixing to the osseous bodies in questions can be visualized only by way of profile radiographs.

Consequently, it appeared that said osteosynthesis plates could be improved, and it is for this reason that the inventors of the present invention have themselves also recently proposed a novel osteosynthesis implant which, among other advantages, has the characteristic of radiolucency, by which it is possible to effectively respond to the aforementioned limitations.

However, it has been observed that such an osteosynthesis implant, however advantageous it may be in respect of the problems mentioned above, does not offer the surgeon the possibility of precise monitoring, other than visual, of the positioning of said implant opposite a given anatomical zone, for example the “watershed” line of a radius.

In particular, the surgeon is unable to monitor this relative positioning with the aid of traditional X-rays, since the osteosynthesis implant is not visible upon radiography.

The invention therefore aims to remedy this technical shortcoming and to make available a novel shoe which serves for helping to position and fix a surgical implant and which is able to be easily attached to said implant in such a way as to permit precise monitoring of the position of said implant with respect to said osseous bodies of the patient, even when said implant is not itself directly visible by radiography.

It is another object of the invention to make available a novel shoe and a novel surgical kit associated therewith, making it possible to facilitate the operation of positioning and fixing the implant in the body of the patient, by virtue of a better appreciation of the position of the surgical implant.

It is another object of the invention to make available a novel shoe and a novel surgical kit associated therewith, permitting better monitoring of the development of the fracture of a bone of a patient.

It is another object of the invention to make available a novel shoe and a novel surgical kit associated therewith, permitting rapid and practical implementation in the context of a surgical operation.

It is another object of the invention to make available a novel shoe and a novel surgical kit associated therewith, of which the design and manufacture are relatively simple and inexpensive.

It is another object of the invention to make available a novel shoe and a novel surgical kit associated therewith, making it possible to limit the post-operative complications and the pain suffered by the patient.

The objects of the invention are achieved with the aid of a shoe for helping to position and fix a surgical implant which is intended to be attached to osseous bodies of a patient, for example those of the forearm, said shoe having a main body and being characterized in that said main body has:
at least one radiolucent zone and at least one radiopaque zone, and
means for positioning said shoe with respect to the surgical implant in a predetermined relative position,

so as to allow monitoring of the positioning of said implant with respect to said osseous bodies of the patient.

The objects of the invention are also achieved with the aid of a surgical kit including a surgical implant which is intended to be attached to osseous bodies of a patient, for example those of the forearm, and a shoe according to the above description.

Other objects and advantages of the invention will become clearer on reading the following description and by referring to the attached drawings which are provided purely for illustration and are non-limiting, and in which:

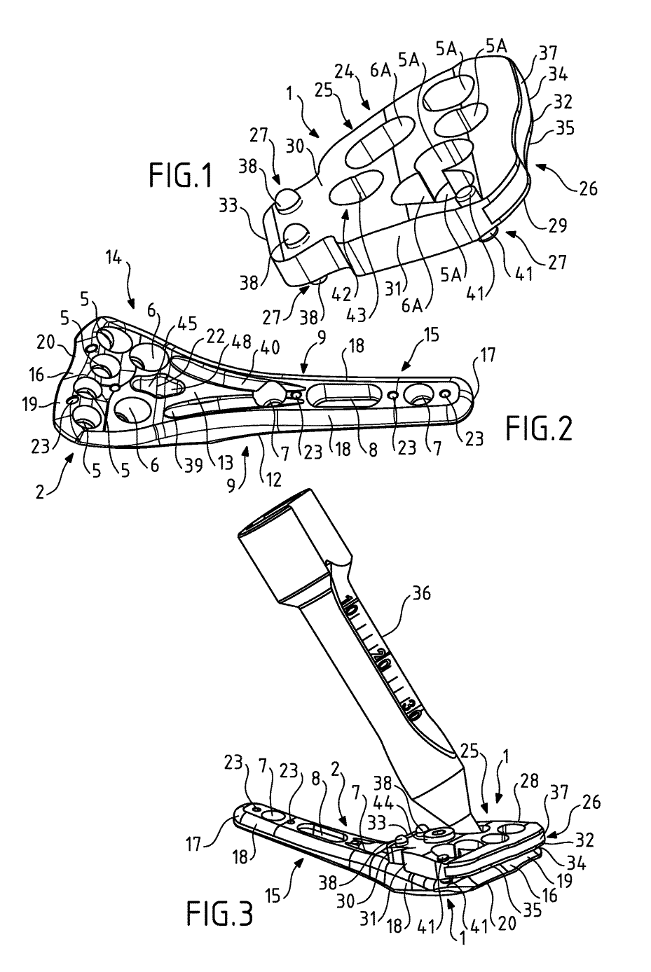

FIG. 1 shows a general perspective view of a shoe for helping to position and fix a surgical implant according to the invention;

FIG. 2 shows a longitudinal perspective view of a preferred embodiment of an osteosynthesis implant able to cooperate with the shoe of the invention;

FIG. 3 shows a general perspective view of the shoe from FIG. 1, attached and fixed to an osteosynthesis implant and combined with a drill guide with a view to preparing to fix said osteosynthesis implant on osseous bodies of a patient;

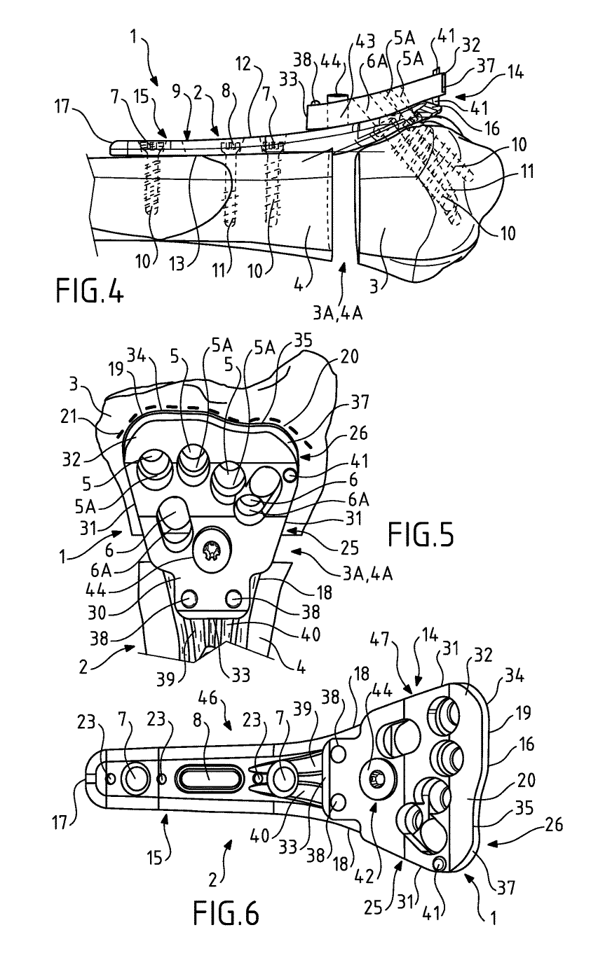

FIG. 4 shows a side view of the general arrangement of a shoe according to the invention, depicted very schematically, on an osteosynthesis implant, which is itself attached and held fixed to osseous bodies in the area of a fracture zone with the aid of fixation screws;

FIG. 5 shows a front view of the shoe from FIG. 1 when attached and fixed to an osteosynthesis implant, the whole arrangement being positioned on a distal radius opposite the “watershed” line of the latter;

FIG. 6 shows a plan view of the shoe from FIG. 1 combined with an osteosynthesis implant, so as to form a surgical kit according to the invention.

The invention relates as such to a shoe 1 for helping to position and fix a surgical implant 2 which is intended to be attached to osseous bodies 3, 4 of a patient, a non-limiting illustrative embodiment of which shoe 1 is depicted in FIG. 1.

Said surgical implant 2 is, for example, intended to be attached to osseous bodies of a forearm and/or the wrist of a patient. In this example, said surgical implant 2 thus forms a forearm implant, specifically a distal radial plate for treating a fracture of the distal end of the radius of the forearm of a patient.

However, the invention is not limited to a shoe 1 for helping to position and fix a forearm implant and will be able to be applied, for example, to a tibial implant or a palmar implant.

In any case, the surgical implant 2, which the shoe 1 according to the invention will help to position and fix, is provided for implantation in the body of the patient during a surgical operation performed, for example, under anaesthesia, in particular following damage such as a fracture or break sustained by a bone of the patient, in order to manage or help manage said damage during an osteosynthesis process. The patient designated by the invention is preferably a human being, although there is nothing to oppose said implant 2 being introduced into and fixed in the body of an animal with the aid of the shoe 1 according to the invention in order to manage similar veterinary pathologies corresponding to the human pathologies presently described.

The surgical implant 2, which the shoe 1 of the invention helps to position and fix, is intended to be attached to osseous bodies 3, 4 of a patient, for example those of the forearm and/or of the wrist, by way of fixation means 5, 6, 7, 8, so as to hold said osseous bodies 3, 4 in position with respect to each other, as is illustrated in FIG. 4 for example, in order in particular to permit, or at least promote, the osteosynthesis thereof.

The surgical implant 2 is thus designed to be introduced into the body of a patient and to be fixed temporarily or permanently to the osseous bodies 3, 4 that have been damaged. The “fixation means” are understood as elements which may or may not belong to the surgical implant 2 and which allow the latter to be secured to the osseous bodies 3, 4.

In the case in point, and as is illustrated in FIG. 4, a surgical implant 2 able to cooperate with the shoe 1 of the invention, such that the latter helps position and fix it, has a main body 9, which is intended to be attached to the osseous bodies 3, 4. The fixation means 5, 6, 7, 8 are preferably formed by screwing openings 5, 6, 7, 8 in which fixation screws 10, 11 (visible in FIG. 4) are intended to be inserted in order to secure said surgical implant 2, in particular the main body 9 thereof, to the osseous bodies 3, 4, by screwing said fixation screws into the latter.

“Osseous bodies” 3, 4 are understood as bones, cartilage, fragments of bones or of cartilage, even tendons, or a combination of these, preferably intended to be fused by osteosynthesis with the aid of the surgical implant 2 which the shoe 1 of the invention helps to position and fix, for example two fragments of one and the same bone that have fractured and been separated by a fracture line 3A, 4A.

As is illustrated in FIG. 4, the main body 9 of the surgical implant 2 advantageously has an anterior surface 12 intended to bear at least partially on said osseous bodies 3, 4, and a free surface 13 arranged opposite the anterior surface 12 and forming a dorsal surface which is preferably free when the surgical implant 2 is in place on said osseous bodies 3, 4.

The following description and the drawings preferably concern a surgical implant in the form of a distal radial plate, which is intended for the treatment of a fracture of the distal end of the radius of the forearm of a patient, and they therefore concern a shoe for helping to specifically position and fix this type of implant.

However, the shoe 1 of the invention may be intended for other types of osteosynthesis implants and will therefore be able to have a design different from that explained below. Without departing from the scope of the invention, it will of course also be able to be used in combination with any type of implant 2 intended to be attached to osseous bodies 3, 4 of a patient.

The main body 9 of a surgical implant 2 in the form of a distal radial plate is preferably composed of a distal plate 14 which is continued by a proximal stem 15.

Said distal plate 14 forms a portion of the main body 9 preferably situated at a first end of the latter, the proximal stem 15 forming the other end in the continuity of said distal plate 14. The main body 9 preferably extends longitudinally between:
a distal edge 16, starting from which the distal plate 14 extends, and
a proximal edge 17, starting from which the proximal stem 15 extends, and transversely between two lateral edges 18.

The distal plate 14 is preferably designed and configured to be attached to a preferably distal osseous body, that is to say situated in front of or anterior to the osseous bodies 3, 4, preferably to a distal part of the bone of the radius, that is to say close to the wrist of the patient.

According to the embodiment illustrated in the figures, this distal plate 14 has a substantially trapezoidal shape, while the proximal stem 15 has a substantially elongate shape, such that the implant 2 has the general shape of a spatula.

The distal edge 16 of the surgical implant 2, which the shoe 1 of the invention helps to position and fix, advantageously has a medial portion 19 and a lateral portion 20, the latter being set back in relation to said medial portion 19, so as to be able to match the anatomical line of the osseous bodies 3, 4, in particular a bone of the radius of the patient.

Preferably, the distal edge 16 extends transversely and is composed of two successive portions, the medial portion 19 being further forward than the lateral portion 20. Thus, the distal edge 16 advantageously has an “S” shape in order to limit the pain suffered by the patient.

Matching the anatomical line of the osseous bodies 3, 4, this “S” shape in fact advantageously corresponds to the route of the crest line of the palmar part of the radius, or the “watershed” line 21, visible in FIG. 5 in the form of a broken line.

The “medial portion” is understood as the part of the distal edge 16 intended to be placed preferably to the medial side of the radius. The lateral portion 20 is preferably intended to be placed to the radial side of the forearm, opposite the medial side.

Thus, the surgical implant 2 on which the shoe 1 of the invention is intended to be positioned can be provided in two principal variants formed by an implant for the left forearm (not shown in the figures) and an implant for the right forearm (shown in the figures).

Preferably, the main body 9 of the implant 2 is provided with distal screwing openings 5 and with at least one proximal screwing opening 7, which are formed in the distal plate 14 and in the proximal stem 15, respectively, and which constitute or contribute to constituting fixation means 5, 6, 7, 8, by way of which the implant 2 keeps said osseous bodies 3, 4 in position with respect to each other. The screwing openings 5, 7 are preferably intended each to receive a fixation screw 10, 11 for fixing the surgical implant 2 to the osseous bodies 3, 4, as can be seen in particular in FIG. 4.

Preferably, the main body 9 of the surgical implant 2 is also provided with a through-opening 22 which is not intended to receive a fixation screw and which is formed through said main body 9 in the area of the zone that interconnects the distal plate 14 and the proximal stem 15.

As is illustrated in particular in FIG. 2, the surgical implant 2 preferably also comprises continuous guide holes 23 formed in the main body 9, the surgical implant 2 being intended to be engaged by way of said guide holes 23 on guide pins (not shown) during its placement in the body of the patient.

During the placement of the surgical implant 2 in the body of the patient, the shoe 1 is intended to be arranged temporarily on the free surface 13 of said implant 2, this free surface 13 corresponding to a surface opposite the anterior surface 12 of the implant 2, the latter surface being intended to bear at least partially on said osseous bodies 3, 4.

According to an important feature of the invention, the shoe 1 has a main body 24 which itself has at least one radiolucent zone 25 and at least one radiopaque zone 26, and means 27 for positioning said shoe 1 with respect to the surgical implant 2 in a predetermined relative position, so as to allow monitoring of the positioning of said implant 2 with respect to said osseous bodies 3, 4 of the patient.

The general principle of the invention in fact lies in a shoe 1 intended to be attached to an implant 2 in at least one known and perfectly controlled position, by way of said positioning means 27 (which will be explained further below), said shoe 1 having at least one radiopaque zone 26 which, during radiographic monitoring, acts as a visible marker of the positioning of said implant 2 with respect to said osseous bodies 3, 4, said marker thus being advantageously offset from and removable from the implant 2.

The shoe 1 of the invention is used to greatest advantage when the implant 2 to which it is attached is completely radiolucent. In this preferred case, the radiopaque zone 26 of the shoe 1 thus advantageously constitutes the only means of marking the positioning of said implant 2.

However, the invention does not exclude the shoe 1 of the invention being used in combination with an implant 2 which is not radiolucent but which, for example, is radiolucent only in the zone of the implant 2 on which the shoe 1 is positioned, or with an implant 2 which is completely radiopaque. In such an extreme configuration, the radiopaque zone 26 of the shoe 1 would then not constitute the sole means, but at least one means of marking the positioning of said implant 2.

Preferably, the main body 24 of the shoe 1 has a base 28 intended to bear on said surgical implant 2.

The base 28 advantageously has a lower surface 29 intended to bear at least partially on the free surface 13 of the implant 2, and an upper surface 30 which is opposite and substantially parallel to the lower surface 29 and forms a preferably free dorsal surface when the shoe 1 is in place on said surgical implant 2.

This base 28 has lateral edges 31, a distal edge 32 and a proximal edge 33. Said base 28 thus extends longitudinally between the distal edge 32 and the proximal edge 33 and transversely between two lateral edges 31.

The lateral edges 31, the distal edge 32 and the proximal edge 33 advantageously delimit a contour corresponding substantially to at least part of the contour of said implant 2 on which the shoe 1 is intended to be positioned.

In particular, according to the preferred embodiment illustrated in the figures, the shoe 1 is intended to be positioned exclusively on the distal plate 14 of the implant 2.

Thus, as is illustrated in FIG. 1, the distal edge 32 of the shoe 1 advantageously has a first portion 34 and a second portion 35, the latter being set back in relation to said first portion 34. Preferably, the distal edge 32 extends transversely and is composed of two successive portions, the first portion 34 being further forward than the second portion 35. The distal edge 32 thus advantageously has an “S” shape. The shape of the distal edge 32 thus advantageously corresponds to the “S” shape of the distal edge 16 of the implant 2.

Moreover, the lateral edges 31 are preferably substantially straight, and said lateral edges 31, the distal edge 32 and the proximal edge 33 are configured in such a way that the base 28 has a substantially trapezoidal contour, thus corresponding to the contour of the distal plate 14 of the implant 2.

According to the preferred embodiment illustrated in the figures, in particular in FIG. 1, the base 28 has, in the area of its distal edge 32, a zone whose thickness decreases preferably gently from the middle part of the base 28 towards said distal edge 32. In addition, and still according to this preferred embodiment, the proximal edge 33 forms an overhang protruding from the generally trapezoidal shape of said base 28.

However, it is alternatively conceivable that the shoe 1 covers the whole of the surface of the main body 9 of said implant 2, in which case the base 28 will in the same way be able to have a contour corresponding to the spatula-shaped contour of said implant 2.

The base 28 of the shoe 1 advantageously has oblong drilling openings 5A, 6A. When the shoe 1 is placed in position on the implant 2, these oblong drilling openings 5A, 6A have the purpose, on the one hand, and in an initial stage, of receiving a drilling guide 36 (shown in FIG. 3) for drilling the bone constituting the osseous bodies 3, 4, while angularly limiting the positioning of said drilling guide 36.

Indeed, said oblong drilling openings 5A, 6A are configured, in terms of their respective orientation, to ultimately allow drilling of the bone by means of the drilling guide 36 only in a given and precise direction.

On the other hand, and in a second stage once the bone has thus been drilled in a desired orientation and the drilling guide 36 removed, and with the shoe 1 being kept in position on the implant 2, said oblong drilling openings 5A, 6A advantageously make it possible to guide the surgeon's positioning and screwing of the fixation screws 10, 11 for fixing the implant 2 on the osseous bodies 3, 4.

The placement of said fixation screws 10, 11 is therefore particularly simple and precise.

As is illustrated in FIG. 1, some oblong drilling openings 5A and 6A can intersect in order to further facilitate the use of the drilling guide 36 and of the fixation screws 10, 11 in a potentially complex and confined operating site.

In the same way, the base 28 of the shoe 1 is advantageously provided with guide holes (not shown) which are intended to be engaged on guide pins (not shown).

The main body 24 of the shoe 1 has, according to the invention, and as mentioned beforehand, at least one radiolucent zone 25, that is to say invisible upon radiography, and at least one radiopaque zone 26, that is to say, on the contrary, visible upon radiography.

According to a first preferred variant of the invention, shown in the figures and in particular in FIG. 1, the base 28 is a one-piece component made from a substantially radiolucent material, which is able to at least partially form said at least one radiolucent zone 25 of the main body 24.

In other words, the base 28 thus constitutes a one-piece component made from a material substantially invisible upon radiography, for example polyether ether ketone (PEEK) or another biocompatible plastic material. Such a base 28 can be easily produced, for example by machining or by injection.

For its part, said at least one radiopaque zone 26 of the shoe 1 according to this first variant is advantageously formed by a substantially radiopaque element 37, separate from said base 28 and fixed to the main body 24.

Such an element 37 is preferably single and in one piece, as shown in the preferred embodiment illustrated in FIG. 1. Said element 37 is produced, for example by machining or by forming, from a material that is substantially visible upon radiography, for example a metallic material based on stainless steel, titanium or another metal or alloy of biocompatible metals.

Alternatively, it is entirely conceivable, without departing from the scope of the invention, that said radiopaque zone 26 is composed of a plurality of elements 37 made from a material that is substantially visible upon radiography.

Said radiopaque element 37 is advantageously fixed firmly and permanently to the base 28 of the shoe 1, for example by said element 37 being fitted into the base 28 or by adhesively bonding said element 37 onto the base 28 with the aid of a suitable glue.

This preferred variant of the invention has the advantage of being very simple to implement, since it entails a relatively simple mechanical combination of materials which are commercially available and whose safe use in the context of intracorporeal therapy has been widely tested.

In a second variant of the invention, not shown in the figures, said base 28 is for the most part made from a substantially radiolucent material which is able to at least partially form said at least one radiolucent zone 25 of the main body 24 of the shoe 1, said material having locally, within its mass, one or more radiopaque zones which are able to locally form at least in part said at least one radiopaque zone 26 of the main body 24.

In this case, the base 28 is thus a single-piece component made from a material or from a mixture of materials which are generally radiolucent but have an appreciable local variation in their radiolucency properties, so as to generate locally one or more zones that are identifiable upon radiography.

Such a composite material can, for example, be produced by implantation of radiopaque particles in a radiolucent matrix or else by local modulation of the density of a substantially radiolucent material, the one or more zones of greater density then having a relative radiopacity contrasting with the zones of lesser density.

The substantially radiopaque element 37 of the preferred variant of the invention is preferably positioned in the area of the distal edge 32 of the base 25 of the shoe 1. In this preferred case, the radiopaque element 37 is thus positioned in contact with the distal edge 32 and is thus, for example, positioned on, against or even inside the distal edge 32 of the base 28.

However, without departing from the scope of the invention, it is conceivable that said radiopaque zone 26 is not positioned in the area of the distal edge 32 of the base 25 of the shoe 1 but instead, for example, in the area of another part of the shoe 1.

Still more preferably, the substantially radiopaque element 37 is positioned so as to match the contour of the distal edge 32 of said base 28, that is to say the element 37 integrates perfectly in the contour of the distal edge 32 and, in particular, does not extend beyond, on the one hand, the lower surface 29 and, on the other hand, the upper surface 30 of the base 28.

Still more preferably, said substantially radiopaque element 37 has a profile identical to that of the distal edge 32 of said base 28 and thus very faithfully reproduces the variations of the profile of the distal edge 32.

Thus, during radiography, the radiopaque element 37 permits clear and very precise identification of the distal edge 32 of the base 28 of the shoe 1 even when said base 28 remains invisible to radiography.

Still without departing from the scope of the invention, it is of course also entirely conceivable that said substantially radiopaque element 37 is positioned and configured differently from the preferred arrangement described above. For example, it will be possible for said substantially radiopaque element 37 to have a different profile from that of the distal edge 32 of said base 28 or else to be positioned slightly in front of, or by contrast set back from, said distal edge 32.

In the particularly advantageous embodiment shown in the figures, the radiopaque element 37 is in the form of a plate, or of a band, engaged in the distal edge 32 of the base 28 of the shoe 1 so as to lie flush with the surface of said distal edge 32, thus very faithfully reproducing the variations of the profile thereof. Alternatively, said radiopaque element 37 could equally well be in the form of a wire engaged in the same way.

During the radiography examination, when taking an X-ray from above, that is to say an X-ray taken in a direction perpendicular to the surface of the implant 2 on which the shoe 1 is positioned, the radiopaque element 37 appears, according to this preferred embodiment, by projection in the plane of the radiography, in the form of a line contrasting with the rest of the elements visible upon radiography.

This line is all the more visible and recognizable when the implant 2 is made of a radiolucent material. It appears in fact as a black line, or at least a darker line, on the X-ray. However, it remains perfectly identifiable when the implant 2 is on the contrary made from a radiopaque material, if the radiopaque material forming the implant 2 is not strictly identical to the material forming the radiopaque element 37.

Assuming an implant 2 made from a substantially radiopaque material, it would be expedient to use, for production of the radiopaque zone 26, a material of significantly lesser or greater density, or a method by which the material used can be given a significantly lesser or greater density than that of the material forming the implant 2, so as to cause the line corresponding to the radiopaque zone 26 to appear by contrast.

The main body 24 of the shoe 1 also has, according to the invention, and as mentioned above, means 27 for positioning said shoe 1 with respect to the surgical implant 2 in at least one predetermined relative position.

Within the meaning of the invention, said predetermined relative position advantageously corresponds to a position in which the shoe 1 is placed on the free surface 13 of the surgical implant 2, such that the distal edge 32 and the lateral edges 31 of the base 28 of said shoe 1 take up a position substantially in line with the distal edge 16 and the lateral edges 18, respectively, of the distal plate 14 of the surgical implant 2.

In this predetermined relative position, the drilling openings 5A, 6A of the shoe 1 are superposed on and advantageously coincident with the screwing openings 5, 6 of the surgical implant 2 when the shoe 1 is attached to the latter.

In the same way, in said predetermined relative position, the guide holes (not shown) of the shoe 1 are advantageously coincident with the guide holes 23 of the surgical implant 2, in such a way that the guide holes of the shoe 1 can be engaged on the guide pins (not shown) by being superposed on the guide holes 23 of the surgical implant 2 when said shoe 1 is positioned on said implant 2.

In this way, when the shoe 1 is attached to the surgical implant 2 and when the latter is positioned with the aid of the guide pins (not shown) on the osseous bodies 3, 4 of the patient, it is possible to carry out drilling or preliminary drilling of the osseous bodies 3, 4, for example with the aid of the drilling guide 36, with a view to inserting the fixation screws 10, 11. As the preliminary drilling or the drilling is carried out through the drilling openings 5A, 6A of the shoe 1, the integrity of the screwing openings 5, 6 is preserved, and the operation of fitting the surgical implant 2 in place is made easier.

However, it is still entirely conceivable, without departing from the scope of the invention, that said predetermined relative position corresponds to a position which differs from the one explained above. For example, provision can be made that, in said predetermined relative position, the shoe 1 is on the contrary placed on the free surface 13 of the surgical implant 2 such that the distal edge 32 and the lateral edges 31 of the base 28 of said shoe 1 are positioned, respectively, set back from or projecting from the distal edge 16 and the lateral edges 18 of the distal plate 14 of the surgical implant 2.

Whatever the case, said predetermined relative position of the shoe 1 with respect to the surgical implant 2 is preferably defined in advance, for example depending on the nature and type of the implant 2 in question, and said positioning means 27 are advantageously designed in such a way that said shoe 1 easily adopts this predetermined relative position, preferably in a “natural” and intuitive manner, when it is attached to the implant 2. However, these positioning means 27 as such preferably do not constitute means for fixing the shoe 1 to the implant 2 to which it is attached, and they are therefore separate from and independent of such fixation means.

Particularly advantageously, these positioning means 27 can constitute error prevention means which prevent or at least substantially limit the risk of the shoe 1 being positioned with respect to the implant 2 in a position other than said predetermined position.

In order to facilitate the placement of the shoe 1 on the implant 2 in said predetermined relative position, the lower surface 29 of the base 28 of the shoe 1, intended to come into contact with the implant 2, is preferably provided with at least two stubs 38 protruding from the lower surface 29 of said base 28.

According to this preferred embodiment, these stubs 38 form, or thus advantageously contribute to forming, said positioning means 27.

As is illustrated in FIG. 1, these stubs 38 are preferably arranged in the area of the proximal edge 33 of the base 28, symmetrically on each side of the longitudinal axis of said base 28.

Said stubs 38 are preferably integral with the base 28 so as to form a one-piece component.

They are advantageously intended to cooperate with at least two longitudinal guide members 39, 40 preferably formed in the free surface 13 of the implant 2, so as to permit the positioning of said shoe 1 with respect to said implant 2 in said predetermined relative position.

According to the preferred embodiment shown in particular in FIG. 2, said longitudinal guide members 39, 40 are formed by two longitudinal grooves 39, 40 formed in the area of the zone of the main body 9 that connects the distal plate 14 to the proximal stem 15 of the implant 2. The longitudinal grooves 39, 40 are preferably arranged in a “V” shape parallel to the lateral edges 18 of the implant 2, the opening of the “V” being oriented in the direction of the distal edge 16 of the implant 2.

The presence of these two stubs 38 and their cooperation with the longitudinal guide members 39, 40 of the implant 2 advantageously facilitate the precise positioning of the shoe 1 on the implant 2, for example by sliding of said stubs 38 in said longitudinal guide members 39, 40, while preventing any movement of rotation of said shoe 1 on said implant 2.

Preferably, said at least two stubs 38 protrude on each side of the lower 29 and upper 30 surfaces of said base 28, such that the shoe 1 can be positioned reversibly on the implant 2 and, according to two variants, thereby constitute a shoe 1 for a right-side implant or for a left-side implant.

In the preferred embodiment shown in the figures, according to which the implant 2 constitutes a distal radial plate, the shoe 1 can therefore be a shoe 1 for a right-side forearm implant, as shown in the figures, or a shoe for a left-side forearm implant, by simply turning the shoe 1 about with respect to the free surface 13 of the implant 2.

According to the preferred embodiment shown in the figures, the lower surface 29 of the base 28 of the shoe 1 is additionally provided with at least a third stub 41. Also protruding from the lower surface 29 of the base 28, this third stud 41 is positioned near the distal edge 32 of said base 28, so as to bear on the distal edge 16 of the implant 2 when the shoe 1 is in place on said implant 2.

Thus, and in particular in the case where the distal plate 14 of the implant 2 has a chamfer in the area of its distal edge 16, this third stub 41 advantageously allows the implant 2 to take up the stress exerted by the surgeon on the shoe 1, thus contributing to the precision of the manoeuvre by which the implant 2 is placed and fixed and to the good mechanical strength of the shoe 1, by avoiding the phenomena of canting.

Similarly to the configuration of the two stubs 38, this third stub 41 also advantageously protrudes, according to the preferred embodiment shown in the figures, on each side of the lower 29 and upper 30 surfaces of said base 28, such that the shoe 1 can be positioned reversibly on the implant 2.

Preferably, the shoe 1 is designed such that said at least one radiopaque zone 26 of the main body 24 has a configuration which imitates that of an anatomical zone, for example the given “watershed” line 21 opposite which said radiopaque zone 26 is intended to be positioned.

In other words, the shoe 1 is advantageously designed such that the profile of the radiopaque zone 26 of its base 28 faithfully follows the profile of an anatomical zone of particular interest of the osseous bodies 3, 4, such that the surgeon is able, during radiography, to precisely monitor the positioning of the implant 2 with respect to this anatomical zone by superposing the line, formed by the projection of the radiopaque zone 26, on the profile of the anatomical zone concerned.

Still more preferably, according to the embodiment illustrated in the figures, said anatomical zone is the “watershed” line 21 of a radius.

In fact, in the particularly advantageous embodiment illustrated in particular in FIG. 4, when the shoe 1 is attached to said surgical implant 2 and positioned in said predefined position with the aid of the positioning means 27, the base 24 of the shoe 1 is preferably positioned on the distal plate 14 of said implant 2 such that, in particular, the distal edge 32 of the shoe 1 takes up a position precisely in line with the distal edge 16 of the implant 2.

Since said at least one radiopaque zone 26 of the main body 24 of the shoe 1 advantageously permits the clear and very precise identification of the distal edge 32 of the base 28 of the shoe 1 upon radiography, then, for this reason, said at least one radiopaque zone 26 is situated, by fitting of the shoe 1 on the implant 2, shaped and configured so as to indirectly permit the monitoring of the positioning of said implant 2, and more particularly the distal edge 16 thereof, with respect to a given anatomical zone of the body of the patient, in the area of which said implant 2 is fixed.

According to this particularly advantageous embodiment, the distal edge 16 of the implant 2 preferably has, as described above, an “S” shape which, by matching the anatomical line of the osseous bodies 3, 4, advantageously corresponds to the route of the crest line of the palmar part of the radius, or the “watershed” line.

Thus, by fitting the shoe 1 on the implant 2 in the predetermined relative position, the radiopaque zone 26 of the shoe 1 advantageously imitates the configuration of this preferably considered anatomical zone, represented by the “watershed” line 21, as is illustrated in FIG. 5.

Said at least one radiopaque zone 26, preferably embodied by this radiopaque element 37, thus makes it possible to monitor the position of the implant 2 with respect to the “watershed” line 21 of the radius on which the implant 2 is intended to be fixed, as is illustrated in FIG. 5.

By performing radiography and simultaneously visualizing, on the one hand, this “watershed” line 21 and, on the other hand, the projection, in the radiography plane, of the positon of the radiopaque element 37 which reflects that of the distal edge 16 of the implant 2, the surgeon can position the implant 2 very precisely on the osseous bodies 3, 4, while ensuring that said distal edge 16 does not protrude beyond said “watershed” line 21.

Alternatively, it is of course entirely conceivable, without departing from the scope of the invention, that the radiopaque zone 26 of the base 28 of the shoe 1 is shaped and configured differently in order to imitate the configuration of another anatomical zone of interest and to permit, with the aid of the shoe 1, the precise positioning of the implant 2 with respect to this other anatomical zone.

Preferably, the main body 24 of the shoe 1 additionally has a securing means 42 by which said shoe 1, in said predetermined relative position, is secured with respect to said surgical implant 2.

This securing means 42 advantageously allows the shoe 1 to be held firmly but removably in position on the surgical implant 2, so as to permit the precise guiding of the drilling guide 36, without the risk that the stress applied by the surgeon to said drilling guide 36 inadvertently causes the detachment of the shoe 1 from the implant 2.

Preferably, this securing means 42 is composed of a securing orifice 43 which extends through the base 28 of the main body 24, said securing orifice 43 being intended to receive a screw 44 for securing the shoe 1 on the surgical implant 2.

According to the preferred embodiment illustrated in the figures, in particular in FIG. 1, this securing orifice 43 is formed in the base 28 of the shoe 1 so as to be closer to the proximal edge 33 than the distal edge 32.

Thus, when the shoe 1 is positioned on the implant 2 in the predetermined relative position defined above, said securing orifice 43 advantageously coincides with the through-opening 22 of said implant 2, said through-opening 22 thus advantageously constituting a receiving element 45 intended to cooperate with said securing means 42 of said shoe 1 by virtue of the securing screw 44, so as to allow the shoe 1 to be secured to the implant 2.

Still more preferably, said securing screw 44 is a screw which allows the shoe 1 to be locked on the surgical implant 2 by rotation of said screw 44 through only a quarter of a turn.

The surgeon is thus able to lock the shoe 1 in position on the surgical implant 2 in a way that is particularly practical, rapid and safe.

Alternatively, without departing from the scope of the invention, the securing means 42 can be composed of a securing screw 44 integral with said main body 24, that is to say able to slide or turn in said securing orifice 43, while remaining mechanically connected to the main body 24.

This variant (not shown in the figures) has the advantage of substantially limiting the risk of loss of the securing screw 44, in particular in the body of the patient, during removal of the shoe 1 from the implant 2. However, in such a configuration, the shoe 1 no longer has the reversibility which is described above and which allows it to be combined, by simple turning, with an implant 2 for the right forearm or an implant 2 for the left forearm.

It is also entirely conceivable, without departing from the scope of the invention, that the securing screw 44 is replaced by another fixation system of the quarter turn type, or else by a fixation system not based on a quarter turn.

The invention also relates to a surgical kit 46, as illustrated in particular in FIG. 6, which includes a surgical implant 2, which is intended to be attached to osseous bodies 3, 4 of a patient, for example those of the forearm, and, on the other hand, a shoe 1 in accordance with the above description.

Preferably, and in accordance with the example described above and illustrated in the figures, the implant 2 included in the surgical kit 46 is an osteosynthesis implant and, more particularly, a distal radial plate, that is to say intended for the treatment of a fracture of the distal end of the radius of the forearm of a patient.

Thus, by correlation, the shoe 1 of the surgical kit 46 is a shoe adapted for helping to position and fix an osteosynthesis implant in the form of a distal radial plate.

However, since the invention is not limited to a shoe 1 for helping to position and fix a forearm implant, it will be possible, for example, for the surgical kit 46 to include a tibial implant or a palmar implant, and their corresponding shoe.

Said surgical implant 2 of the surgical kit 46 preferably has a receiving element 45 intended to cooperate with said securing means 42 of said shoe 1 and at least one radiolucent part 47 so as to permit, on the one hand, the fixing of said shoe 1 on said surgical implant 2 in said predetermined relative position and, on the other hand, the monitoring of the positioning of said implant 2 with respect to said osseous bodies 3, 4.

In fact, as has been mentioned above, the shoe 1 of the invention is used to its greatest advantage when the implant 2 to which it is attached is completely radiolucent or at least partially radiolucent.

Still more preferably, the main body 9 of the surgical implant 2, and more generally the surgical implant 2, will advantageously be able to form a one-piece component made from a radiolucent material, preferably polyether ether ketone (PEEK).

Preferably, the surgical kit 46 includes a shoe 1 in which the lower surface 29 of the base 28, intended to come into contact with the implant 2, is provided with at least two stubs 38 which protrude from the lower surface 29 of said base 28 and are intended to cooperate with at least two longitudinal guide members 39, 40, preferably formed by longitudinal grooves 39, 40, which are formed in the surface of said implant 2, in order to permit the positioning of said shoe 1 with respect to said implant 2 in said predetermined relative position.

Still more preferably, the surgical kit 46 includes the above-described shoe 1 in its variant according to which the base 28 of said shoe 1 has at least two stubs 38 which protrude on each side of the lower 29 and upper 30 surfaces of said base 28, such that the shoe 1 can be positioned reversibly and thus constitute a shoe for a left-side implant or a shoe for a right-side implant.

In the same way, the shoe 1 included in the surgical kit 46 is advantageously in its preferred variant according to which its base 28 has the third stub 41, the latter also advantageously protruding, in the preferred embodiment shown in the figures, on each side of the lower 29 and upper 30 surfaces of said base 28.

This advantageously makes it possible to simplify the stocking and preparation of the surgical kits to be marketed, since there is then just one standard shoe 1 to be taken into account for each of the right-side or left-side variants of an implant 2.

When the shoe 1 of the invention is attached to the surgical implant 2 of the surgical kit 46 in said predetermined relative position, said at least one radiolucent zone 25 of the main body 24 of the shoe 1 is advantageously intended to lie opposite at least one radiolucent part 47 of said implant 2.

Still more preferably, in said predetermined relative position of the shoe 1 on the implant 2, the distal edge 32 of the base 28 of the shoe 1 is intended to lie substantially opposite a distal end of said surgical implant 2.

In the preferred embodiment illustrated in the figures, according to which the implant 2 is a distal radial plate, the distal edge 32 of the base 28 of the shoe 1 will thus be positioned opposite the distal plate 14 of said implant 2, preferably in line with the distal edge 16 of the implant 2.

Thus, said shoe 1 and implant 2 of the surgical kit 46 are preferably configured in relation to each other such that the shoe 1 is used to its greatest advantage, i.e. by virtue of its radiopaque zone 26 it constitutes a particularly useful marker of the precise position of the implant 2, and more precisely of the distal edge 16 of the implant 2, with respect to the osseous bodies 3, 4 of the patient.

According to the above preferred description of said shoe 1 and implant 2, when said shoe 1 of the kit 46 is attached to a surgical implant 2 of said kit 46, said at least one radiopaque zone 26 of the main body 24 of the shoe 1 is advantageously shaped and configured to permit the monitoring of the positioning of said implant 2 with respect to a given anatomical zone, more particularly with respect to the “watershed” line 21 of a radius, the configuration of said “watershed” line 21 being advantageously imitated by the radiopaque zone 34, for the reasons set out above.

In addition, said receiving element 45 of said implant 2 is preferably composed of an orifice 48 intended to cooperate with said securing screw 41 of said shoe 1 in order to allow said shoe 1 to be locked with respect to said implant 2 in said predetermined relative position, which orifice 48 is moreover not intended for fixing said implant to said osseous bodies 3, 4.

In a preferred embodiment of the invention, this receiving element 45 is composed of the through-opening 22 of the implant 2 in accordance with the preferred description thereof given above.

Consequently, the combination of the shoe 1 of the invention with a surgical implant 2, according to the preferred description thereof which is given above and which is illustrated in the figures, constitutes a surgical kit 46 which is particularly of practical use for the surgeon and allows him to fully tackle the problems associated with the precise positioning of the distal edge 16 of the implant 2 with respect to the “watershed” line 21 of the treated radius, in the particular case where the implant 2 is radiolucent, or when at least its distal plate 14 is radiolucent.

Particularly advantageously, it will also be possible for the surgical kit 46 to include the fixation screws 10, 11 for fixing the shoe 1 on the implant 2, guide pins, and also the screw 44 for securing the shoe 1 in position on said implant 2, the whole assembly being preferably packed in a sterile packaging.

Moreover, in order to make the intervention easier for the surgeon, and in particular to reduce the length of the operation for fixing the implant 2 in place in the body of the patient, the surgical kit 46 will also advantageously be able to include the shoe 1 of the invention already positioned and secured on said implant 2 in said predetermined relative position.

Finally, the invention will also relate as such to a method for fitting a surgical implant 2 in place with the aid of the shoe 1 of the above-described surgical kit 46. The above description concerning the shoe 1, the surgical implant 2 and the surgical kit 46 is also applicable to the positioning method explained below.

The method for fitting the surgical implant 2 in place with the aid of the shoe 1 of the invention advantageously comprises at least the following successive steps:
an incision is made in the patient's body so as to be able to introduce the surgical implant 2 into the inside, in the area of the osseous bodies 3, 4 to be treated,
guide pins are implanted in the osseous bodies 3, 4, for example by impaction, and optionally with the aid of the surgical implant 2 or the shoe 1, it being possible for the guide pins (not shown) to be impacted into the osseous bodies 3, 4 through guide holes 22 in these,
the surgical implant 2 is positioned on the osseous bodies 3, 4 with the aid of the guide pins, in such a way that the anterior surface 12 of the implant 2 bears at least partially on the osseous bodies 3, 4, the distal plate 14 of the implant 2 being in contact with an end part of the bone to be treated forming the osseous body 3, and the proximal stem 15 being in contact with an intermediate or middle part of the bone to be treated forming the osseous body 4,
the shoe 1 is attached to the free surface 13 of the surgical implant 2 by preferably sliding the stubs 38 in the longitudinal guide members 39, 40 formed in said free surface 13 of the surgical implant 2, so as to bring the distal edge 32 of the base 28 of the shoe 1 in line with the distal edge 16 of the distal plate 14 of the implant 2 and the distal screwing openings 5, 6 of said distal plate 14 in line with the drilling openings 5A, 6A of the shoe 1,
the shoe 1 is secured in position on the surgical implant 2 with the aid of the securing screw 44,
radiography is performed to verify, by visualization of the radiopaque zone 26 of the shoe 1, the correct positioning of the surgical implant 2 on the osseous bodies 3, 4, with respect to the fracture zone 3A, 4A to be treated and its anatomical environment,
preliminary drilling of the osseous bodies 3, 4 is performed through all or some of the drilling openings 5A, 6A of the shoe 1 and through the openings 7, 8 of the proximal stem 15 of the implant 2,
a first clamping screw 10 is inserted into one of the distal screwing openings 5 of the distal plate 14 of the implant 2, and a second clamping screw 11 is inserted into the screwing opening 8 of the proximal stem 15,
before tightening said first and second clamping screws 10, 11, the proximity and the orientation of the osseous bodies 3, 4 are adjusted by sliding the second clamping screw 11 along the screwing opening 8,
the first and second clamping screws 10, 11 are tightened,
the fixation screws 9 are then inserted into all or some of the remaining screwing openings 5, 6, 7, in order to ensure stable fixing of the surgical implant 2 in the body of the patient,
as the operations of inserting and clamping the fixation screws 10, 11 proceed, the guide pins are removed from the osseous bodies 3, 4,
radiography is performed in order to verify the orientation of the screws 10, 11 through the osseous bodies 3, 4,
the shoe 1 is unlocked and removed from the surgical implant 2,
the incision in the patient's body is closed.

Of course, it will be possible for some steps to be changed around without departing from the scope of the invention. In particular, the step of fitting the shoe 1 on the implant 2 can be carried out before inserting said implant 2 into the body of the patient, so as to make the surgeon's work easier.

Although a surgical implant 2 in the form of an osteosynthesis plate has preferably been considered above by way of example and in detail, the positioning method according to the invention applies more generally to any type of implant 2 intended to be attached to osseous bodies 3, 4 of a patient.

Thus, more generally, the method for fitting a surgical implant 2 in place with the aid of a shoe 1 from a surgical kit 46 according to the invention comprises at least the following steps, of which the order of implementation may optionally be changed around without thereby departing from the scope of the invention:
the shoe 1 is attached to the surgical implant 2 in a predetermined relative position with the aid of the means 27 for positioning of said shoe 1;
an incision is made in the patient's body so as to be able to introduce the surgical implant 2 into the inside, in the area of the osseous bodies 3, 4 to be treated;
the surgical implant 2 is positioned with respect to the osseous bodies 3, 4;
radiography is performed in order to verify, by visualization of the radiopaque zone 26 of the shoe 1, the correct positioning of the surgical implant 2 with respect to the osseous bodies 3, 4.

In conclusion, the shoe 1 allows the surgical implant 2 to be fitted in place easily, precisely and rapidly, thereby helping to permit particularly rapid and high-quality osteosynthesis of the osseous bodies 3, 4, while limiting the post-operative complications and the pain suffered by the patient.

What is claimed is:

1- Shoe (1) for helping to position and fix a surgical implant (2) which is intended to be attached to osseous bodies (3, 4) of a patient, for example those of the forearm, said shoe (1) having a main body (24) and being characterized in that said main body (24) has:
at least one radiolucent zone (25) and at least one radiopaque zone (26), and
means (27) for positioning said shoe (1) with respect to the surgical implant (2) in a predetermined relative position,
so as to allow monitoring of the positioning of said implant (2) with respect to said osseous bodies (3, 4) of the patient.

2- Shoe (1) according to the preceding claim, characterized in that the main body (24) has a base (28) intended to bear on said surgical implant (2) and having lateral edges (31), a distal edge (32) and a proximal edge (33), which delimit a contour corresponding substantially to at least part of the contour of said implant (2).

3- Shoe (1) according to claim 2, characterized in that said base (28) is a one-piece component made from a substantially radiolucent material and is able to at least partially form said at least one radiolucent zone (25) of the main body (24), said at least one radiopaque zone (26) being formed by a substantially radiopaque element (37) which is separate from said base (28) and is fixed to the main body (24).

4- Shoe (1) according to claim 3, characterized in that said substantially radiopaque element (37) is arranged in the area of the distal edge (32) of said base (28).

5- Shoe (1) according to the preceding claim, characterized in that said substantially radiopaque element (37) matches the contour of the distal edge (32) of said base (28).

6- Shoe (1) according to the preceding claim, characterized in that said substantially radiopaque element (37) has a profile identical to that of the distal edge (32) of said base (28).

7- Shoe (1) according to claim 2, characterized in that said base (28) is for the most part made from a substantially radiolucent material which is able to at least partially form said at least one radiolucent zone (25) of the main body (24), said material having locally within its mass one or more radiopaque zones which are able, locally, to at least partially form said at least one radiopaque zone (26) of the main body (24).

8- Shoe (1) according to any one of the preceding claims, characterized in that said at least one radiopaque zone (26) of the main body (24) has a configuration which imitates that of a given anatomical zone opposite which said radiopaque zone (26) is intended to be positioned.

9- Shoe (1) according to the preceding claim, characterized in that said at least one radiopaque zone (26) of the main body (24) has a configuration which imitates that of the “watershed” line (21) of a radius.

10- Shoe (1) according to any one of the preceding claims, characterized in that the main body (24) additionally has a means (42) by which, in said predetermined relative position, said shoe (1) is secured with respect to said surgical implant (2).

11- Shoe (1) according to the preceding claim, characterized in that said securing means (42) is composed of a securing orifice (43) passing through the base (28) of the main body (24), said securing orifice (43) being intended to receive a screw (44) for securing the shoe (1) on the surgical implant (2).

12- Shoe (1) according to the preceding claim, characterized in that said securing screw (44) is a screw (44) which allows the shoe (1) to be locked on the surgical implant (2) by a rotation of said screw (44) through only a quarter of a turn.

13- Surgical kit (46) including a surgical implant (2) intended to be attached to osseous bodies (3, 4) of a patient, for example those of the forearm, and also a shoe (1) according to one of the preceding claims.

14- Surgical kit (46) according to the preceding claim, characterized in that the surgical implant (2) has a receiving element (45) intended to cooperate with said securing means (42) of said shoe (1) and at least one radiolucent part (47), so as to permit, on the one hand, the fixation of said shoe (1) on said surgical implant (2) in said predetermined relative position and, on the other hand, the monitoring of the positioning of said implant (2) with respect to said osseous bodies (3, 4) of the patient.

15- Surgical kit (46) according to the preceding claim, characterized in that, with said shoe (1) attached to said surgical implant (2) in said predetermined relative position, said at least one radiolucent zone (25) of the main body (24) lies opposite at least one radiolucent part (47) of said implant (2).

16- Surgical kit (46) according to the preceding claim, characterized in that the distal edge (32) of the base (28) of the shoe (1) lies substantially opposite a distal end (14) of said surgical implant (2).

17- Surgical kit (46) according to any one of claims 13 to 16, characterized in that the lower surface (29) of the base (28) of the shoe (1), intended to come into contact with the implant (2), is provided with at least two stubs (38) which project from the lower surface (29) of said base (28) and which are intended to cooperate with at least two longitudinal guide members (39, 40) formed in the surface (13) of said implant (2) in order to permit the positioning of said shoe (1) with respect to said implant (2) in said predetermined relative position.

18- Surgical kit (46) according to the preceding claim, characterized in that said at least two stubs (38) project from each side of the lower (29) and upper (30) surfaces of said base (28), such that the shoe (1) can be positioned reversibly and thus constitute a shoe for a left-side implant or a shoe for a right-side implant.

19- Surgical kit (46) according to one of claims 13 to 18, characterized in that said receiving element (45) of said implant (2) is formed by an orifice (48) intended to cooperate with said screw (44) for securing said shoe (1), in order to allow said shoe (1) to be locked with respect to said implant (2) in said predetermined relative position, which orifice (48), moreover, is not intended to fix said implant (2) on said osseous bodies (3, 4).

20- Surgical kit (46) according to any one of claims 13 to 19, characterized in that said surgical implant (2) is a distal radial plate.

21- Surgical kit (46) according to the preceding claim, characterized in that, with said shoe (1) attached to a surgical implant (2), said at least one radiopaque zone (26) of the main body (24) is shaped and configured so as to permit monitoring of the positioning of said implant (2) with respect to the “watershed” line (21) of a radius, of which the configuration is imitated by the radiopaque zone (26).

22- Method for fitting a surgical implant (2) in place with the aid of a shoe (1) from a surgical kit (46) according to any one of claims 13 to 21, characterized in that said method comprises at least the following steps:
the shoe (1) is attached to the surgical implant (2) in a predetermined relative position, with the aid of the means (27) for positioning said shoe (1);
an incision is made in the body of the patient in order to be able to introduce the surgical implant (2) into the inside, in the area of the osseous bodies (3, 4) to be treated;
the surgical implant (2) is positioned with respect to the osseous bodies (3, 4);
radiography is performed to verify, by visualization of the radiopaque zone (26) of the shoe (1), the correct positioning of the surgical implant (2) with respect to the osseous bodies (3, 4).
